(19) United States
(12) Patent Application Publication
(10) Pub. No.: US 2017/0095417 A1
(43) Pub. Date: Apr. 6, 2017

(54) Percutaneous Implement and Method for Manufacturing

(71) Applicants: Yoshikazu Tobinaga, Shiga (JP); Yutaka Hara, Tokyo (JP)

(72) Inventors: Yoshikazu Tobinaga, Shiga (JP); Yutaka Hara, Tokyo (JP)

(21) Appl. No.: 14/876,794

(22) Filed: Oct. 6, 2015

(51) Int. Cl. A61K 9/00; A61K 31/375; A61K 8/67; A61K 47/26; A61K 8/60; A61K 8/73; A61K 31/167; A61K 38/28; A61K 31/5575; A61K 8/65; A61K 8/29; A61K 8/19; A61Q 19/02; A61Q 19/00; A61M 37/00; A61K 8/02

(52) U.S. Cl. CPC A61K 9/0021; A61K 8/0216; A61K 31/375; A61K 8/676; A61K 47/26; A61K 8/60; A61K 8/735; A61K 31/167; A61K 38/28; A61K 31/5575; A61K 8/65; A61K 8/29; A61K 8/19; A61Q 19/02; A61Q 19/00; A61M 37/0015; A61M 2207/10; A61M 2037/0046; A61M 2037/0053

(57) ABSTRACT

Micro implement with micro payload is used for painless and safe percutaneous drug delivery. The micro•head made of maltose or saccharide contains drug and stands on micro-stem installed in orifice of stem holder. Non drug micro-stem supports the payload to adjust insertion depth and reach an interest region under skin and is set to come out from the orifice just after insertion. The payload is completely bullet-like inserted into skin and the drug contained micro•head in the upper micro payload is also delivered into skin completely. The bioavailability 100% is accomplished. The inserted payload solves in internal water and releases drug around the interest region. The implement is composed of payload and holder. The payload is unified of micro•head with drug and non-drug micro•stem. All saccharides means ecologically disposable.

### BACKGROUND

The present invention relates to percutaneous technologies for medications, cosmetics, material implantation for beauty, nutrition for health, drug delivery for deep organs under skin, and medical devices and microneedle for dermal administration as the technologies in common, and also relates to a 3 dimensional molding micro processing technology for mounting a microneedle to a holder by registration controlled precisely in 0.001 mm ground rule, and including fine transportation technology dealing with small drug chip or micro objects to manufacture the devices.

Applicant(s) hereby incorporate herein by reference any and all patents and published patent applications cited or referred to in this application.

By way of background, microneedle technology for transdermal drug delivery has been highly expected. The object is not restricted only to skin disease treatment, beauty treatment and beauty operation but also painless or non-invasive medical devices for treatment of immune system disease relating to regions in the depths of skin and entire body, but the technology is actually behind the expectation because the progress of relevant technology is stagnating for uncertainty factors due to product and manufacturing technology. Simple and convenient microneedle aiming for a high level of drug delivery effectiveness is highly expected, and its product having function of drug delivery absolutely allowed to reach inside the body with repeatable and reliable needle part of accurate form and volume are highly expected.

Exactly dosing medical devices have been highly expected, and conventional manufacturing technologies of microneedles accommodating to the expectations are resin-treated injecting molding, grinding work and beam processing such as laser beam or synchrotron radiation X-rays, but the common feature is restricted to the deformation processing of the substrate or the materials. Microneedles applied to conventional micro machine of less than 0.1 mm length mostly comes from substrate process which is restricted to the method influenced by material characteristics of substrate like integrated electronic circuit. The method could end up in different feature far from requirement property. The following reference shows a representative example of microneedles originated from the substrate deformation, but the quantitative dosage rate and convenience was imperfect.

Subject on reliability that is quantitativity including bio availability at drug delivery is most important. There are administration methods by means of coat-type microneedles of inorganic or organic materials coating drug on the side or the point, and of mix-type microneedles mixing drug with internal solving materials. The coat-type microneedles can hardly administrate the whole dose due to remove at injection, and the mix-type microneedles can be hardly inserted the whole needle, therefore, a guarantee of dose quantitativity for precise delivery of whole drug is difficult.

In the mix type microneedles, useless drug leaked out to the substrate is avoidable by a means of manufacturing needles separately from substrate and after that the needles are assembled on the substrate. Since errors of being incapable of insertion occur from uneven adhesive strength, a part of drug left on the substrate is wasted. Microneedles in the form of completely removing like a bullet have been sought.

Short needles less than 1 mm have been mainly manufactured because only skin has been targeted for the treatment. Application for the regions in the deep part of the skin or entire body has lately increased including immune system and diabetes. Microneedles longer than 1 mm have been expected for painless administration targeting the deep regions. However, such drug mixed microneedles don't exit.

In microneedle separately manufactured the needle from the substrate, the installing method, which allows to carry and hand such a small object into a holder or a substrate as external diameter less than 1 mm and the length less than 5 mm, is needed. A difficult process that microneedle formed a small and light object around 0.001 mg is independently be conveyed and assembled into a holder is needed. However, there is not micro transportation technology and manufacturing technology to deal with light micro object less than 0.001 mg level.

Drug mixed transdermal microneedles made of degradable materials functioned dissolving the materials and releasing the drug into skin but we had an issue of low bioavailability or unstable quantitativity due to insufficient injection without inserting whole needle up to needle root into skin. To solve the issue, needle tips are expected to definitely be inserted into skin surface and up to the needle root. The perfect insertion is obtained by completely releasing needles from substrate in medication by a structure of different material needle from substrate and automatically detaching needles from a substrate. Then, drug in the needles are expected to be carried into skin completely. However, the structure, with which a uniform size of needles were completely released and drug was delivered into skin with stable quantitativity and repeatability, was not available. Furthermore, the detachable structure, with which had flexibility of needles were steadily fixed before medication and were completely released from substrate, was not obtained. We set subject on seeking the structure of the complete drug delivery function.

We had only short length microneedles of less than 1 mm, since conventional microneedles were targeted only the skin for medication. Recently, the treatment targeting deep region or whole body is much expected especially about immune disease and diabetes and then long microneedles of longer than 1 mm are occasionally wanted to painlessly deliver drug to deep regions. Since such long microneedles with mixed drug were not realized, the function forming long microneedle is sought as a subject.

Registration of fine ground rule at less than 0.01 mm pitch is necessary for assembly of micro head sized from 0.1 mm to 5 mm and micro stem as well. There is not such a fine micro processing method for assembling drug chips or functional drug chips and mechanical structure for the fine assembly and registration are sought as a subject.

Aspects of the present invention fulfill these needs and provide further related advantages as described in the following summary.

### SUMMARY

Aspects of the present invention teach certain benefits in construction and use which give rise to the exemplary advantages described below.

The present invention solves the problems described above by providing a percutaneous implement and method of manufacturing. In at least one embodiment, there is provided a percutaneous micro implement comprising micro payload mounting onto the orifice of a stem holder and having a pyramidal micro-head made from water solvable material including drug with a column micro-stem integrated under the micro-head.

In another aspect, the micro head comprises external tip radius of less than 0.01 mm, the length from 0.02 mm to 5 mm, external radius of the base from 0.01 mm to 1 mm, and any other shaped as any of pyramid, selected from trigonal pyramid, tetragonal pyramid, half shell cone, half shell bullet, cone, bullet or polygonal pyramid.

In another aspect, the micro payload comprises the micro-head in the upper part, and pure micro-stem of the external radius from 0.01 mm to 1 mm and the length from 0.02 mm to 5 mm made from water solvable material with the same shape of the micro-head base in the lower part.

In another aspect, the percutaneous micro implement is characterized by structure having a pyramidal micro-head containing drug chips.

In another aspect, the micro-head structure as assembled comprises the shape of a pure or drug combining two half-shell cones, which is any of a pure cone, a cone containing drug, a combination of pure half-shell cone and half-shell cone containing drug, a combination of drug containing half-shell cone and another drug containing half-shell cone, a quadrangular pyramid comprised of pure pyramid and drug containing pyramid, a quadrangular pyramid comprised of drug containing pyramid and another drug containing pyramid, a combination of drug containing polygonal pyramid and another drug containing polygonal pyramid allowed to be flat on the base when to put together.

In another aspect, the materials of the percutaneous micro implement of drug are characterized by maltose, pullulan, collagen, hyaluronic acid and any of other polysaccharide mentioned in claim 1 and claim 3 as water solvable material, or composite material combined as necessary.

In another aspect, the percutaneous micro implement is characterized by being any of maltose or mixture with maltose mentioned in claim 1 and claim 3 as water solvable material.

In another aspect, the percutaneous micro implement, which is any of vitamins, hyaluronic acid, collagen, protein, DNA, other drugs, nutrient, supplement, cosmetic materials, antibody drug, in vivo specified pigment, in vivo specified metal, in vivo specified metal oxide, in vivo specified magnetic material, biological material, in vivo monitoring material, cell, extra cellular matrix material, or the composite as necessary.

In another aspect, the percutaneous micro implement is characterized by storing any of drug chip, solidified composite material; mixture of maltose or other saccharide with any drug.

In another aspect, the percutaneous micro implement is characterized by the stem holder comprising of maltose, pullulan, hyaluronic acid, cellulose, other saccharide, paper, wood, resin, metal or silk, and having a support with orifice accepting the micro-stem base, or having plural supports arranged in a substrate.

In another aspect, a manufacturing method of the percutaneous micro implement is characterized by using a rectangular-solid micro-mold for the pyramidal micro-head having inverse-shaped pyramidal dent coincident with the mold in planes and having one or more dent or recess on the rectangular solid, to form a pyramidal and a half-shell pyramidal micro-head with a half-shell cone and a half-shell bullet.

In another aspect, a manufacturing method of the percutaneous micro implement is characterized by a micro-mold for micro-stem with inverse-shaped prism dent or prism recess to fit the micro stem shape in the surface of the rectangular solid forming the micro stem in any shape of triangular prism, quadrangular prism, column, half-shell column.

In another aspect, a manufacturing method of the percutaneous micro implement is characterized by mounting a micro payload, integrated by cooling coagulation or drying solidification, into the stem holder orifice by applying pressure or ultrasonic vibration to form drug material with thermo-plasticity or moist plasticity into a pyramidal or cone shape integrated by three parts of a micro mold for micro head, a micro mold for micro stem and a holder.

In another aspect, a manufacturing method of the percutaneous micro implement is characterized by having a simultaneous three dimensional mobile function finely adjustable in the three directions of back and forth, right and left, and up and down and releasing gradually at speed pitch of 0.001 mm per second vertical to the micro mold surface, in the fine releasing process of the molds from formed object of the micro-head and the micro-stem.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1A is a perspective schematic view of an exemplary micro implement apparatus, in accordance with at least one embodiment;

FIG. 1B is a perspective schematic view of the micro payload thereof, in accordance with at least one embodiment;

FIG. 2A is a side schematic view thereof, in accordance with at least one embodiment;

FIG. 2B is an exploded side schematic view thereof, in accordance with at least one embodiment;

FIGS. 3A-3C are side schematic views thereof as inserted into the skin in three operational modes, in accordance with at least one embodiment;

FIG. 4A is a side schematic view thereof having an alternate micro stem inserted into the skin, in accordance with at least one embodiment;

FIG. 4B is a side schematic view thereof having a further alternate micro stem inserted into the skin, in accordance with at least one embodiment;

FIG. 5A is a perspective schematic view of an exemplary micro head, in accordance with at least one embodiment;

FIG. 5B is a perspective schematic view of an alternate micro head, in accordance with at least one embodiment;

FIG. 6A is a perspective schematic view of the micro head of FIG. 5A on a micro stem, in accordance with at least one embodiment;

FIG. 6B is a perspective schematic view of the micro head of FIG. 5B on a micro stem, in accordance with at least one embodiment;

FIG. 7A is a perspective schematic view of a further alternate micro head, in accordance with at least one embodiment;

FIG. 7B is a perspective schematic view of a further alternate micro head, in accordance with at least one embodiment;

FIG. 8A is a perspective schematic view of a further alternate micro head, in accordance with at least one embodiment;

FIG. 8B is a perspective schematic view of a further alternate micro head, in accordance with at least one embodiment;

FIG. 8C is a perspective schematic view of the micro head of FIG. 8A on a micro stem, in accordance with at least one embodiment;

FIG. 8D is a perspective schematic view of the micro head of FIG. 8B on a micro stem, in accordance with at least one embodiment;

FIG. 9 is a perspective schematic view of a further alternate micro head, in accordance with at least one embodiment;

FIG. 10 is a perspective schematic view of a further alternate micro head, in accordance with at least one embodiment;

FIG. 11A is a perspective schematic view of the intersection of three planes, in accordance with at least one embodiment;

FIG. 11B is a perspective schematic view of an exemplary micro mold as resulting from the intersection of three planes illustrated in FIG. 11A, in accordance with at least one embodiment;

FIG. 12A is a perspective schematic view of the micro mold of FIG. 11 B now with an exemplary holder in accordance with at least one embodiment;

FIG. 12B is a perspective schematic view of a further alternate micro head as formed in the micro mold of FIG. 12A, in accordance with at least one embodiment;

FIG. 13 is a perspective schematic view of an alternate micro mold, in accordance with at least one embodiment;

FIG. 14A is a perspective schematic view of a further alternate micro head, in accordance with at least one embodiment;

FIG. 14B is a perspective schematic view of a further alternate micro stem as formed in the micro mold of FIG. 13, in accordance with at least one embodiment;

FIG. 15 is a perspective schematic view of the micro head of FIG. 14A on the micro stem of FIG. 14B, in accordance with at least one embodiment;

FIG. 16 is a perspective schematic view of an alternate micro implement comprising the micro head of FIG. 14A and the micro stem of FIG. 14B as assembled as shown in FIG. 15, in accordance with at least one embodiment;

FIG. 17 is a perspective schematic view of a further alternate micro implement comprising the micro head of FIG. 14A and an alternate micro stem, in accordance with at least one embodiment;

FIG. 18 is a perspective schematic view of a further exemplary micro implement and micro mold, in accordance with at least one embodiment;

FIG. 19 is a perspective schematic view of a further exemplary micro implement, in accordance with at least one embodiment; and

FIG. 20 is a perspective schematic view of a plurality of exemplary micro implements arranged on a substrate, in accordance with at least one embodiment

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

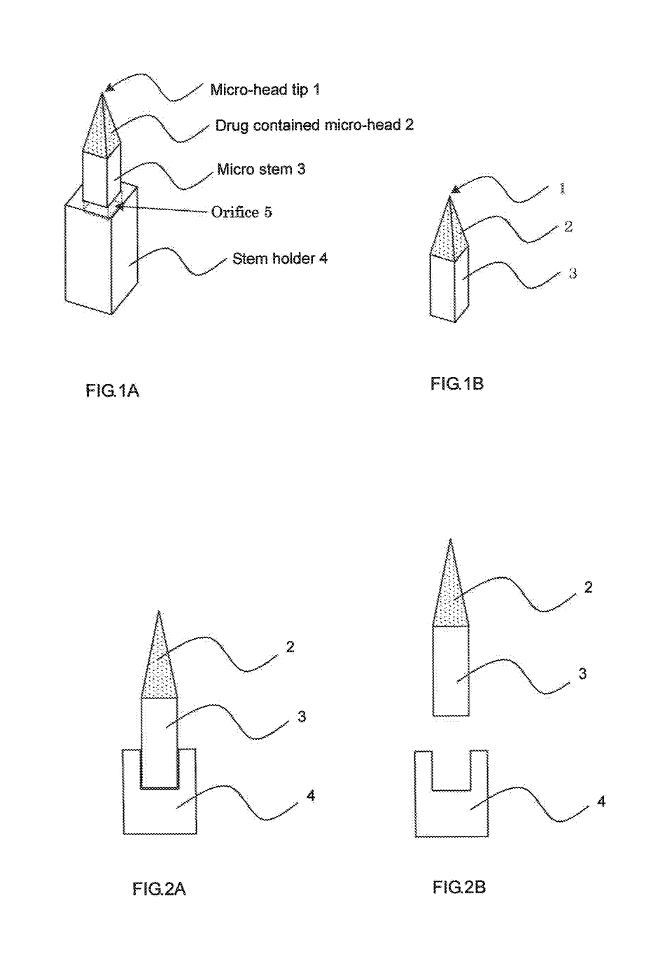

The micro implement as shown in FIG. 1A has structure being installed the micro payload in FIG. 1B by inserting bottom end of the micro payload into the appropriate orifice on an upper surface of the stem holder 4 to fix the micro payload. Since the micro payload is integrated of the micro head 2 having the sharp tip 1 of triangular pyramidal or rectangular pyramidal on top with made from drug mixed saccharide and the micro stem 3 shaped triangular or rectangular column with made from pure saccharide under the micro head, the micro implement cab be released in medication as shown in FIG. 2B. In addition, the micro implement allows to level target depth in skin such as coneeous layer 6, dermal layer 7, and organs under skin 8 by adjusting length of the micro stem. Furthermore, plural pieces of the micro implements are able to stand on a substrate.

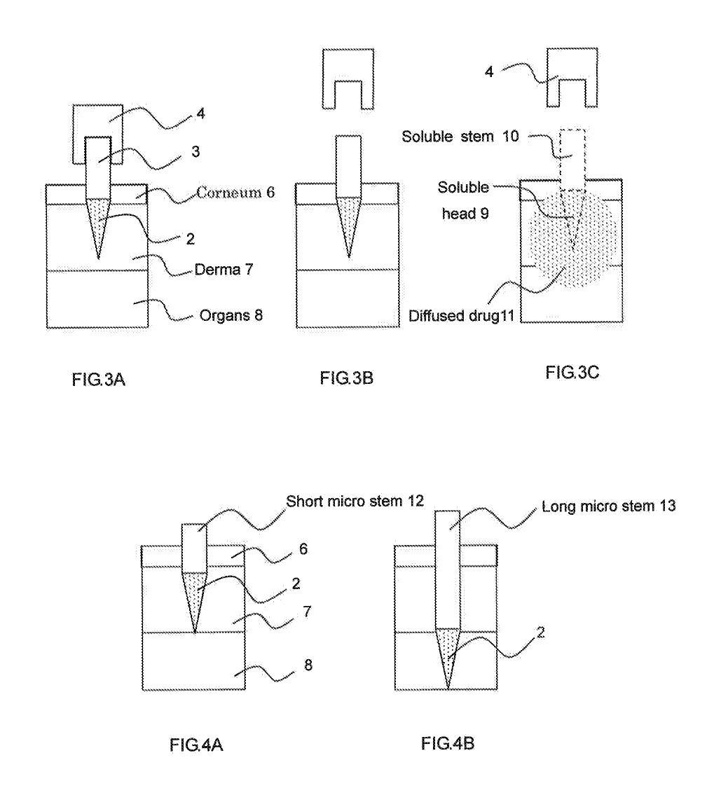

The micro implement installed the micro payload made from maltose or the other saccharide materials allows to painlessly insert into skin and keep inserting to enough as deep level as touching stem holder and then leave only the micro payload in skin by releasing from the stem holder as shown in FIG. 3B, after the micro implement is pulled out from skin at enough insertion as shown in FIG. 3A. Almost all the micro payload is inserted from tip to body in skin at complete insertion, though the bottom end of the micro stem fixed into the orifice of the stem holder is exposed in skin surface. Since the micro payload is constructed of the micro head mixed drug in the upper part and the micro stem in lower part, the micro head is completely inserted in skin. The micro head of the micro payload disappears with dissolving and releasing drug in skin water in a few minutes like 3 minutes after insertion 9, 10 as shown in FIG. 3C, resulting that all drug is diffused in skin to generate infiltration region 11 and 100% bioavailability is achieved as stable drug deliver condition and quantity conservation. Drug delivery specification is clear.

The saccharide micro payload of short micro stem is targeted at only skin as shown in FIG. 4A and the micro payload for deep target as shown in FIG. 4B can deliver drug to deeper region than 1 mm at shallowest. The deep micro payload allows us to treat immune disease with vaccine and diabetes disease needed whole body circulation with insulin. Furthermore, the long micro implement itself becomes volume increase and up to 50 mm cube with expected sufficient amount drug, ranging in delivery amount at mile gram unit weight as equivalent to as treating the immune reaction and the insulin drug.

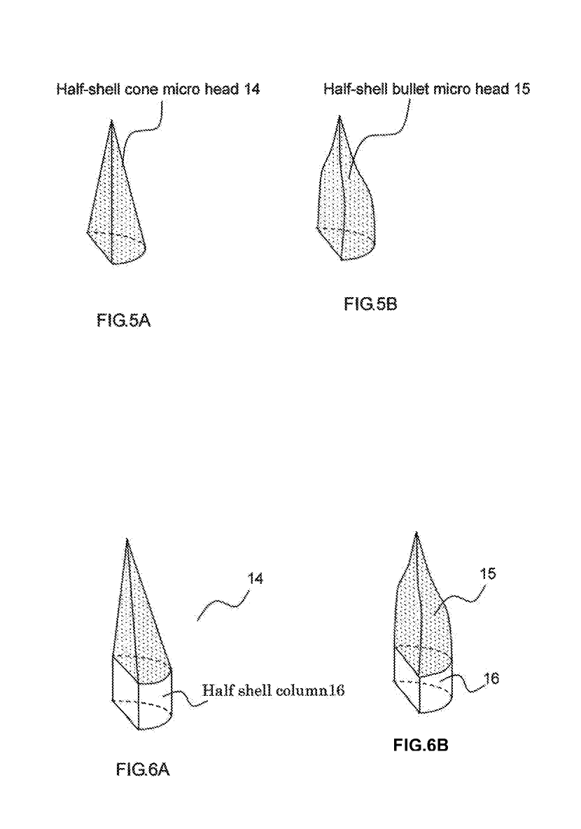

The micro payload integrated of the micro head 2 and the micro stem 3 is formed by the method of forming the micro head from maltose or saccharides in condition of heating melt and cooling harden in the first process with the micro mold for the micro head, the micro stem in the same condition as the micro head in the second process with the micro mold for the micro stem, and by the method of identically connecting the base of the micro head and the upper surface of the micro stem with assembling both the micro mold in highly precise positioning. The formed product of the micro head mixed drug with a sharp tip is obtained by detaching from the molds after the three processes. The half shell cone 14 in FIG. 5A and the half shell bullet 15 in FIG. 5B is formed by the same processes and assembly. Furthermore, the payload product of the half shell cone or half shell bullet is obtained with integrating half shell column 16 by the same process and assembly as shown in FIG. 6A or FIG. 6B respectively. Here, the half shell cone is defined half fragmental solid cut through top of the cone and the half shell bullet is defined as well as the half shell cone.

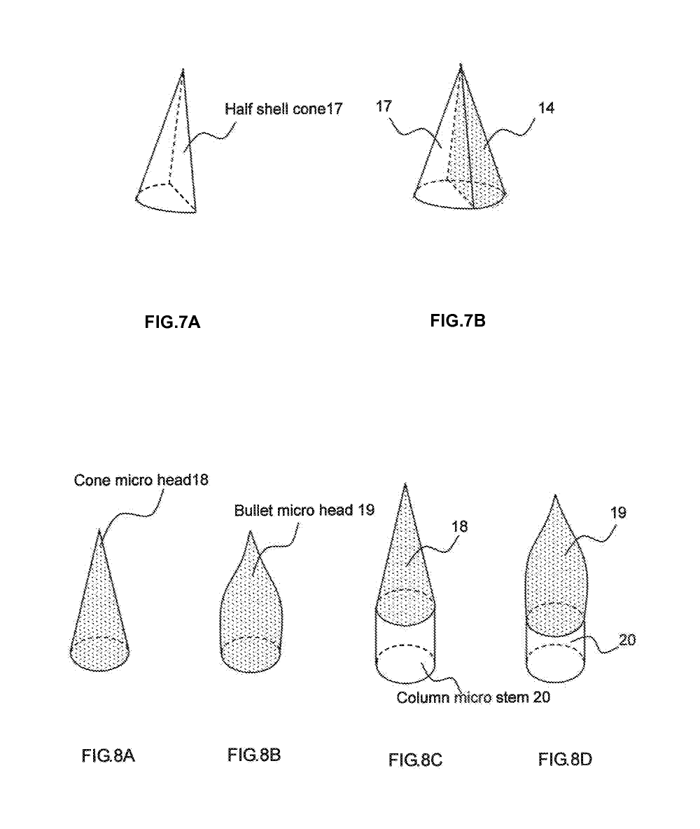

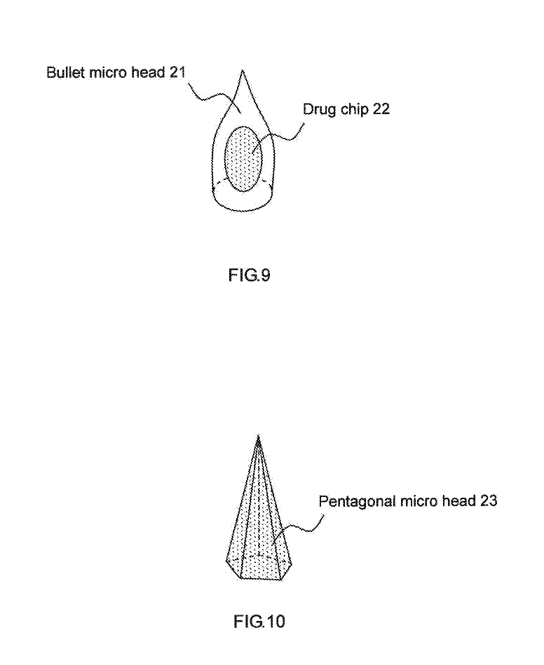

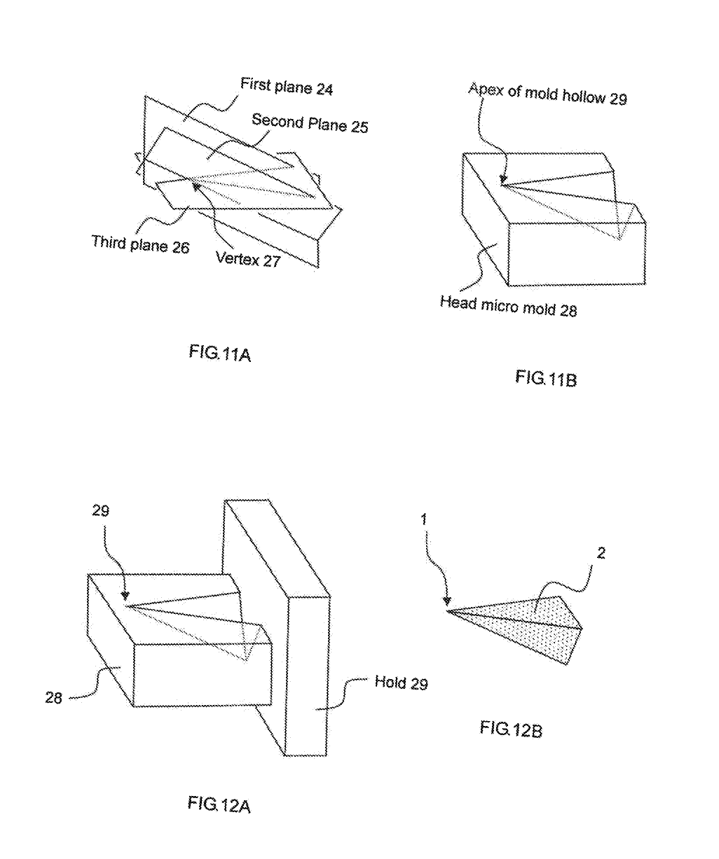

The two half shell cones are assembled to makes a cone. The conical micro head can have a combining solid of the micro head of half shell cone 14 with contained drug and the micro head of half shell cone 17 without drug as shown in FIG. 7. The conical micro head keeps well inserting function supported by half shell cone without drug, even if the half shell cone with drug becomes malfunction due to softening by drug mixture. In the other case, the conical micro head can have two kinds of drugs in the half shell cone with a drug and another half shell cone with another drug. In addition, the conical micro head 18 can duplicate drug amount by assembling two of the same half shell micro head with drug as shown in FIG. 8A and the duplicated bullet shaped micro head 19 is formed as well as shown in FIG. 8B. The micro payload installed the conical micro head with integrated the column micro stem 20 as shown in FIG. 8C. The micro payload installed the bullet shaped micro head is formed as well as shown in FIG. 8D. In extension, the micro payload contained a drug chip inside the bullet shaped micro head is formed as shown in FIG. 9 or becomes the micro the pentagonal pyramidal micro head by combining triangular pyramid and rectangular pyramid as shown in FIG. 10.

In conventional microneedles, needles are directly connected to substrate and needle roots are left after only half tip side of needles are inserted in medication, resulting in bioavailability of less than 70% but more than 30% is lost as waste potion. In the micro implement with preferred embodiment of the invention having the micro payload mixed drug inlayed in the micro stem, drug in the micro payload is completely carried into skin, resulting in 100% bioavailability.

Though conventional transdermal microneedles are unstable in bioavailability due to changeable volume of microneedles inserted into skin every medication, the micro payload with preferred embodiment of the invention is stable because the micro payload is completely released from the stem holder with the micro stem. Then, bioavailability is directly equaled to the volume and shape of the micro payload and is constant every medication with repeatability.

Though conventional microneedles are shorter than 1 mm and do not reach deep region under skin at all, the micro implement with preferred embodiment of the invention installed the micro payload with length of more than 1 mm can be manufactured by the micro mold delivers drug to far deep region under skin and the length itself increases the volume with increasing drug amount, resulting in effective treatment for immune disease and diabetes, which cannot be treated by conventional microneedles.

Conventional microneedle, which is obtained from substrate by processing, is needed to manage waste after medication with heavy works due to residual substrate having residual part of needles. In the micro implement with preferred embodiment of the invention, the micro payload is completely transported into skin with released from the stem holder, leaving only substrate and stem holder after medication. The residual waste, which is substrate of saccharides without touching skin, does not need a special waste management meaning effective cost reduction. In the case of all saccharide stem holder and substrate, waste of the micro implement after medication becomes dissolvable completely, solving environmental issues.

Micro payload installed in the micro implement is an integration of drug mixing amorphous maltose micro head with shaped a variety of pyramids like triangular or quadrangular and adhered on a column micro stem made from pure maltose for depth adjustment. In case of treating shallow region in skin like coneeous layer, micro stem is not needed and the micro head itself works as a micro payload. In deep treatment under skin, the micro payload is an integration of the micro head and the micro stem. The micro head is any pyramidal shape having identical base to the upper surface shape of the micro stem with a sharp tip in less than 0.01 mm at external radius and 0.01 mm to 5 mm length. The micro stem is a column at length from 0.2 mm to 5 mm for the depth adjustment and has sectional external radius from 0.01 mm to 0.5 mm.

In the micro implement form, the micro payload installed into an orifice on the upper surface of the stem holder is integrated a various pyramidal shaped micro head with a sharp tip and a micro stem for depth adjustment, the micro payload functions a microscopic medical device to insert into skin such as microneedles for percutaneous drug delivery. Drug is mixed in amorphous maltose or other saccharide and the micro implement made from the maltose or the saccharide dissolves by the water in skin or body just like a micro drug capsule. The micro payload with installed a drug mixed maltose or saccharide micro head completely comes out from the holder orifice in medication or insertion, staying in skin and then the dissolving micro head releases the drug in the skin by diffusion and infiltration. In any case of vitamins, hyaluronic acid or collagen is selected as a drug, the micro implement, which is integrated amorphous maltose or other saccharide micro head with mixed drug at concentration from 10% to 90% on a pure micro stem installed in the orifice of the stem holder, is manually inserted into skin. Then the micro payload comes out from the holder and the micro head perfectly inserts in the skin like a bullet, allowing the whole drug to stay in skin. The micro head with mixed anything of vitamins, hyaluronic acid or collagen is optimally formulated so that the head dissolves and disappears in body internal water with delivering it into skin at 100% bioavailability.

The micro implement is a dermal drug delivery device characterized by mixing drug in the micro head. The principal ingredient mixing in the maltose micro head are pullulan, polysaccharide, collagen, hyaluronic acid or the complex. The tip of the head sizes less than 0.01 mm at external radius. The length of the head sizes from 0.02 mm to 5 mm. The base of the head sizes external radius from 0.02 mm to 5 mm. The head shapes any pyramid, quadrangular pyramid, a half-shell cone, a half-shell bowling-pin, polygonal pyramid, or a cone or bullet by matching half-shells manufactured with micro molds. Drug chip containing micro head has a form like shown in FIG. 9.

The micro stem, which is the lower part of micro payload of micro implement, is aimed at inserting the micro head in skin deeply and systemic drug like insulin, hormone, or prostaglandin is useable. The material is preferably identical to the micro head so that maltose, pullulan, other saccharide, hyaluronic acid, collagen, or the complex is appropriate. Any other material in vivo affinity dissolved safely in skin like chitin and chitosan, or biodegradable materials like poly lactic acid is usable as the candidate.

Regarding shape of micro-mold of the micro-head, the tip size of the micro-head is the most important for medication or insertion function to deliver percutaneous drug and forming the micro-head mold 28 to sharpen a micro-head tip is important too. Method of sharpening the tip of a pyramidal micro-head is principally optimized to derive the minimum size of intersection 27 from cross cutting among three planes of the first plane 24, the second plane 25 and the third plane 26. The micro head pyramid is a replica released from the identical dint opened in the micro mold. The tip 1 of the pyramid is originated from the tip 29 of the pyramidal dint by the method with a preferred embodiment of the present invention and the dint tip is touched on the upper surface of the mold. There are three planes in the dint of the micro-mold to form the three planes of the pyramid of the micro-head, corresponding the first plane and the second plane of micro-head to two sides in the dint of the micro-mold and corresponding the third plane of the micro-head to open triangle on the upper surface of the micro-mold. The optimized manufacturing method with a preferred embodiment of the present invention is making planarization of melted maltose poured into the dint as the third plane by sweeping to make the third plane and sharpen the tip of the micro-head.

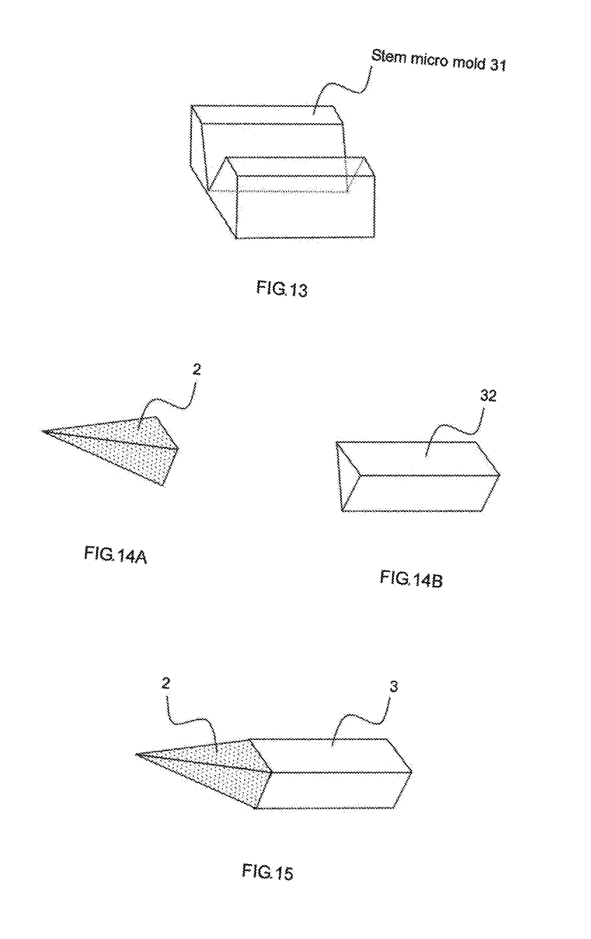

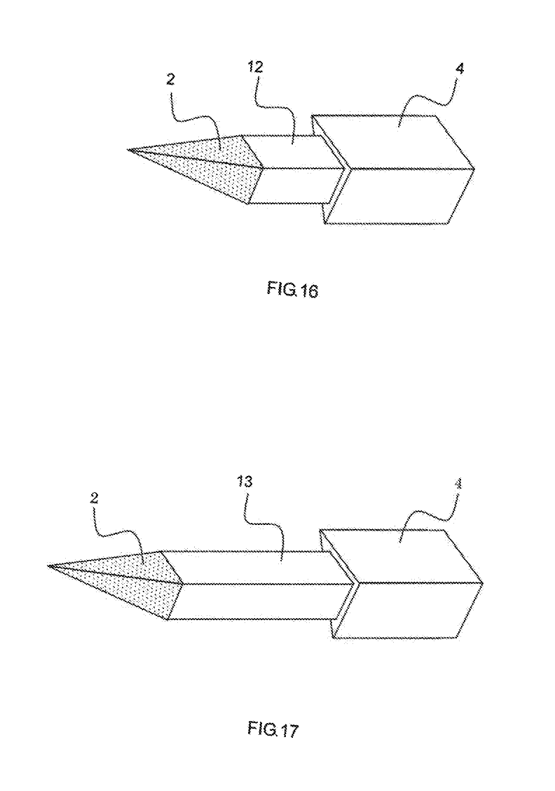

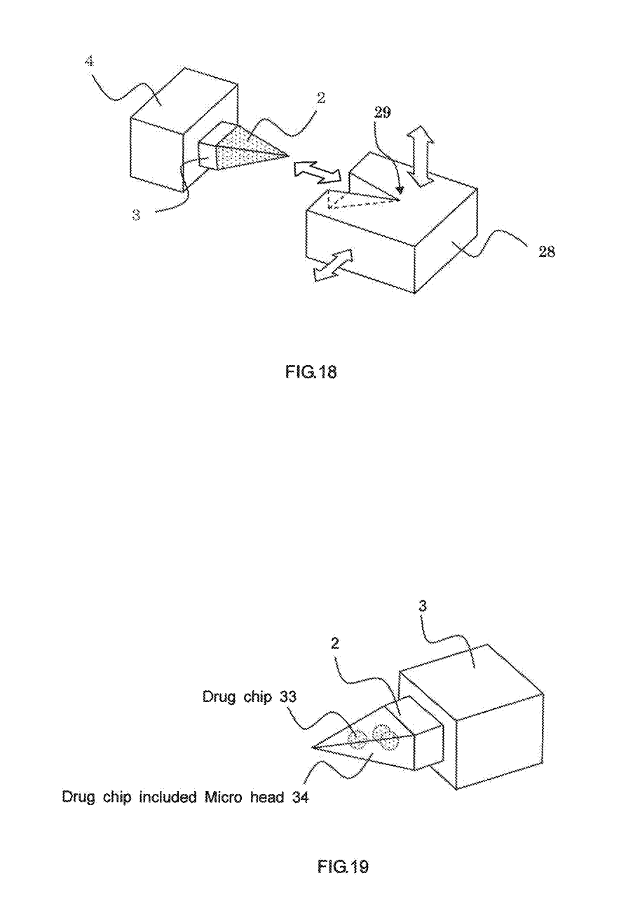

Regarding the micro implement manufacturing method with preferred embodiment of the invention having the micro head or the micro payload with mixed drug, while the case of triangular pyramidal micro head is assumed, the mold 28, having the dint of lied triangular pyramid or more than 2 the pyramidal dints identical to inversed the pyramidal micro head opened in stainless cuboid mold fitting the base arranged onto the mold sidewall and upper surface of pyramidal dint is coincided to the upper surface of the cuboid, is prepared. Drug mixed viscous maltose softened by heating or drug mixed poly saccharides in dilution is poured into the dints in the mold, which has open bases of the triangular pyramidal dints in side wall of the mold and the open bases are closed with the hold 30 attached to the sidewall of the mold. The two surfaces of triangular pyramidal micro head are corresponding to the two surfaces of the pyramidal dint in the mold and the upper surface of the mold head is processed by sweeping over flowing maltose and surface planarization to level the surface at the upper surface of the mold in heating. After processing, the micro head is cooled down in the mold to be hardened and is pulled out from the mold to be formed. In the case of using the micro head as a micro payload without micro steam, the micro head is precisely moved to the lateral direction by 0.001 mm unit digital control to remove and the pyramidal micro payload is made as shown in FIG. 12B. In the case of forming the micro payload integrated the drug mixed micro head and the micro stem, the micro head is held after its formation in the first process and is connected the base of the micro head to the end of the micro stem in the second process with heating or wetting, after the micro stem is formed with the micro mold 31 in FIG. 13 for the micro stem 32 in FIG. 14 in the second process. The micro payload is sequentially is detached with the precise mechanism from the micro mold for micro stem and from the micro mold for micro head. Here, the precise removing is defined as form of micro processing technology which functions to shake up and down, right and left and forth and back in three dimensional degree of freedom in order to detach the micro head from the micro mold with precise adjustment at 0.001 mm pitch. In addition, an easy detachment is obtainable if necessary vibration or ultrasonic waves are applied to both the micro molds by appropriate power ranged from 1 W to 10 W. In addition, the two micro molds as shown in FIG. 18 is allowed to drive with flexibility of three directions by 0.001 mm unit and be heated and cooled simultaneously. Furthermore, the manufacturing method allows to make the micro implement of any long stem like short stem in FIG. 16 or that of long stem in FIG. 17.

The micro implement with preferred embodiment of the present invention allows to include pure solid medicine, complex medicine mixed the material of the micro head, micro capsule included medicine, and micro capsule included micro magnetic granule or micro electromagnetic granule as drug chip 33 in the micro head 34 in FIG. 19. The drug chip forms ellipsoid, rectangular or polygonal pyramid of the material selectable from the following materials: vitamins, collagen, hyaluronic acid, protein, DNA, medical agent, nutrient, supplement, cosmetic material, antibody test agent, pigment, metal, metal oxide, or the mixture, or that of mixture selected from the upper materials in weight ratio from 10% to 90%.

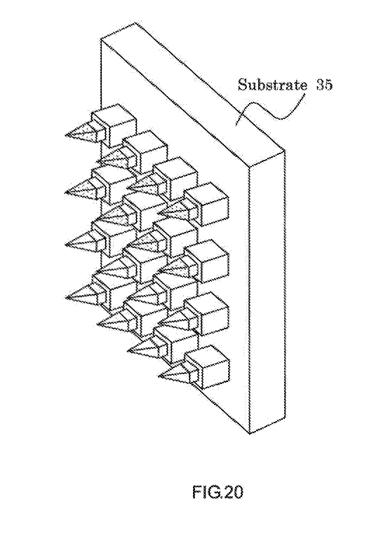

The stem holder, which installs the micro payload in the micro implement with preferred embodiment of the present invention, is fulcrum having orifice inserted the base of the micro payload i.e. the base of the micro stem. The micro stem is stably inlayed onto the orifice of the stem holder in store and has to be completely released from the orifice when the micro payload is inserted into skin. Then, the micro stem is appropriately installed in the stem holder by slightly pushing it into the orifice in 3% smaller than the base of the micro payload with application of vibration or ultrasonic waves ranged in 1 kHz to 10 kHz. In addition, the long stem holder shaped like a stick is easy to handle in the case of a micro payload. Multiple of the micro payloads are lined up in array or a pin support and multiple of the stem holders form standing on the substrate in FIG. 20. Since material of the holder is touched the skin at insertion of the micro payload but is taken off after use, the material doesn't require safety as strictly as in vivo residue. Any of saccharide, paper, wood, resin, metal, maltose, pullulan, hyaluronic acid, cellulose, solid protein, silk or bio material or the complex can be the material. Saccharide, however, is preferable in perspective of reducing the waste disposal cost.

The following non-limiting examples are provided for illustrative purposes only in order to facilitate a more complete understanding of the disclosed subject matter. These examples are intended to be a mere subset of all possible contexts in which the percutaneous implement may be utilized. Thus, these examples should not be construed to limit any of the embodiments described in the present specification, including those pertaining to the various percutaneous implements and/or methods and uses thereof. Accordingly, the practical examples explained hereinafter do not limit the invention.

### EXAMPLE 1

A micro implement was installed a micro payload. The payload was integrated of a 0.003 mm tip sized, 0.7 long and 0.25 mm bottom side quadrangular pyramidal micro-head in up part and a 0.5 mm long and 0.25 mm wide cubic micro-stem in down part. The micro-head is made of amorphous Maltose with mixed 17% Sodium Ascorbic acid and Rhodamine 6B. The micro-stem is made of pure Maltose. In human skin medication by using 100 pieces micro implements arrayed on a substrate by 10 rows and 10 columns, 100% the drug was transferred into skin by recognizing the color of Rhodamine 6B. The bio availability 100% resulted.

### EXAMPLE 2

By using bullet shaped micro-head of 1 mm length and 0.2 mm bottom diameter including two 50 k-100 k molecular weight Hyaluronic acid 0.01 mm side long cubic chips with impregnated Rhodamine 6B, the chip was inserted and completely transferred into skin. Skin surface region around the inserted chip became swollen.

### EXAMPLE 3

Micro implement sample installed micro head with mixed Lidocaine, one of local anesthetic drug, was made by a metal micro mold having 20 hollows of a triangular pyramidal micro-head of 1 mm length in a side line of stainless metal. The sample was used for animal test. Rat was administrated and blood plasma was sampled in leg, arm, stomach, back, and neck, resulting that 0.003-0.01 mg Lidocaine was detected in the plasma.

### EXAMPLE 4

0.01 mg Insulin was obtained in a micro implement installed in a 3 mm stem holder having a micro payload integrated a pyramidal micro head of 3 mm length with mixed 50% Insulin from bovine pancreas in Maltose and 2 mm length Maltose micro-stem. 1 mg Insulin was transferred into body with a 100 micro implements by 10 rows and 10 columns in a medication. 1 mg Insulin is equaled to 26 IU. A diabetes patient who needs 60 IU in a day is three times provided enough in morning, noon and night with 80 micro implements in a cassette, while 60 IU is equivalent to 2.3mg. 1 mg Insulin including micro implement deserves diabetes medical treatment.

### EXAMPLE 5

A sample of micro payload integrated a 1 mm length conic micro-head with drug and 2 mm length micro-stem without drug was injected in rat. The conic micro-head was matched a half-shell Maltose cone mixed 1 mg Alprostadil derived from Prostagladin and a half-shell Maltose cone mixed Rhodamine 6B. The color of Rhodamine 6B was recognized in rat whole body with 20 mmHg hypotension indicating vasodilation from Alprostadil.

### EXAMPLE 6

A sample of 2 mm length micro implement installed a bullet shaped micro-head contained a 2 mg hyaluronic acid chip mixed 1 mg Collagen extracted Collagen solution from human fibroblasts was used for an animal trial with rat. 50% Collagen was transferred into skin. Aggregating Collagen was recognized in intercellular matrix from 1000 times of magnified microscopic view of skin cross section figure.

### EXAMPLE 7

64 micro implements are arranged in a substrate. The micro implement is installed just a half-shell cone micro-head of 0.5 mm length and bottom diameter 0.2 mm made from anhydrous and amorphous Maltose contained 5% cosmetic material of 50% white Titanium oxide and 50% brown iron oxide. Micro implement medication 0.3 mm depth into skin for facial stain displayed stain reduction and brighter skin seemed recognized around the region.

### EXAMPLE 8

Quadrangular pyramidal micro-head of 0.7 mm length and 0.1 mm bottom side length was contained anhydrous and amorphous oblate spheroidal Maltose chip of 0.02 mm length and 0.01 mm diameter. The chip was made from mixing material of 50% Maltose and 50% Hyaluronic acid included 30% albumen which was non-heat resistance limited in 70 degree centigrade. Three micro-heads were installed in three lengths of micro implements of 1 mm, 2 mm, and 3 mm. These were used for animal tests of rat, resulting in albumin aggregation in skin. The three aggregation depths were corresponded to the three length respectively. Albumin resisted temperature of the micro implement forming process.

### EXAMPLE 9

Triangular pyramidal Maltose micro-head of 0.5 mm length and 0.1 mm bottom side length included steel oxide particles in size of average 0.003 mm diameter. The micro-head was installed in 1 mm length micro implement and inserted into skin. 1 mG magnetic field was exposed around the particles inserted skin region. The particles were magnetized. Bioactivity around the region was recognized by detecting the magnetic field originated from the particles in skin.

The micro implement with preferred embodiment of the invention and the manufacturing method are useful in medical treatment and beauty treatment. The method belongs to only low cost mechanical field with processing a sharp tip as fine as 0.001 mm at largest and includes elements of three dimensional process but does not include a fine process of vacuum process and lithography, which have been used as a fine technologies. The method can be carried to mass production.

In closing, regarding the exemplary embodiments of the present invention as shown and described herein, it will be appreciated that a percutaneous implement and method for manufacturing are disclosed. Because the principles of the invention may be practiced in a number of configurations beyond those shown and described, it is to be understood that the invention is not in any way limited by the exemplary embodiments and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A percutaneous micro implement comprising a micro payload mounted onto an orifice of a stem holder and having a pyramidal micro head made from water solvable material including a drug with a column micro stem integrated under the micro head.

2. The micro implement of claim 1 wherein the micro head has an external tip radius of less than 0.01 mm, a length from 0.02 mm to 5 mm, an external radius of the base from 0. 01 mm to 1 mm, and being shaped as a pyramid selected from trigonal pyramid, tetragonal pyramid, half shell cone, half shell bullet, cone, bullet, or polygonal pyramid.

3. The micro implement of claim 2 wherein the micro stem has an external radius from 0.01 mm to 1 mm and a length from 0.02 mm to 5 mm, being made from water solvable material with the same shape as the micro head base in the lower part thereof.

4. The micro implement of claim 3 wherein the water solvable material is selected from maltose, pullulan, collagen, hyaluronic acid or polysaccharide or any combination thereof.

5. The micro implement of claim 3 wherein the water solvable material is selected from maltose or a mixture with maltose.

6. The micro implement of claim 3 comprising any of vitamins, hyaluronic acid, collagen, protein, DNA, other drugs, nutrient, supplement, cosmetic materials, antibody drug, in vivo specified pigment, in vivo specified metal, in vivo specified metal oxide, in vivo specified magnetic material, biological material, in vivo monitoring material, cell, extra cellular matrix material, or any combination thereof.

7. The micro implement of claim 2 comprising any of vitamins, hyaluronic acid, collagen, protein, DNA, other drugs, nutrient, supplement, cosmetic materials, antibody drug, in vivo specified pigment, in vivo specified metal, in vivo specified metal oxide, in vivo specified magnetic material, biological material, in vivo monitoring material, cell, extra cellular matrix material, or any combination thereof.

8. The micro implement of claim 1 comprising any of vitamins, hyaluronic acid, collagen, protein, DNA, other drugs, nutrient, supplement, cosmetic materials, antibody drug, in vivo specified pigment, in vivo specified metal, in vivo specified metal oxide, in vivo specified magnetic material, biological material, in vivo monitoring material, cell, extra cellular matrix material, or any combination thereof.

9. The micro implement of claim 1 wherein the water solvable material is selected from maltose, pullulan, collagen, hyaluronic acid or polysaccharide or any combination thereof.

10. The micro implement of claim 1 wherein the water solvable material is selected from maltose or a mixture with maltose.

11. The micro implement of claim 1 wherein the stem holder comprises one of maltose, pullulan, hyaluronic acid, cellulose, other saccharide, paper, wood, resin, metal or silk, and having a support with orifice accepting a base of the micro stem or having plural supports arranged in a substrate.

12. A method of manufacturing the micro implement according to claim 2 comprising using a rectangular-solid micro-mold for the pyramidal micro head having an inverse-shaped pyramidal dent coincident with the mold in planes and having one or more dents or recesses on the rectangular-solid to form a pyramidal and a half-shell pyramidal micro head with a half-shell cone and a half-shell bullet.

13. A method of manufacturing the micro implement according to claim 3 comprising using a micro-mold for the micro stem with an inverse-shaped prism dent or prism recess to fit the micro stem shape in the surface of a rectangular-solid forming the micro stem in any shape of triangular prism, quadrangular prism, column, or half-shell column.

14. A percutaneous micro implement comprising a pyramidal micro head containing drug chips.

15. The micro implement of claim 14 comprising any of vitamins, hyaluronic acid, collagen, protein, DNA, other drugs, nutrient, supplement, cosmetic materials, antibody drug, in vivo specified pigment, in vivo specified metal, in vivo specified metal oxide, in vivo specified magnetic material, biological material, in vivo monitoring material, cell, extra cellular matrix material, or any combination thereof.

16. The micro implement of claim 14 wherein the drug chips comprise solidified composite material or a mixture of maltose or other saccharide with any drug.

17. A percutaneous micro implement comprising a micro-head structure as assembled having the shape of a pure or a drug combining two half-shell cones, selected from a pure cone, a cone containing a drug, a combination of pure half-shell cone and half-shell cone containing a drug, a combination of a drug containing a half-shell cone and another drug containing a half-shell cone, a quadrangular pyramid comprised of a pure pyramid and a drug containing pyramid, a quadrangular pyramid comprised of a drug containing pyramid and another drug containing pyramid, a combination of a drug containing polygonal pyramid and another drug containing polygonal pyramid configured to be flat on the base when assembled.

18. The micro implement of claim 17 comprising any of vitamins, hyaluronic acid, collagen, protein, DNA, other drugs, nutrient, supplement, cosmetic materials, antibody drug, in vivo specified pigment, in vivo specified metal, in vivo specified metal oxide, in vivo specified magnetic material, biological material, in vivo monitoring material, cell, extra cellular matrix material, or any combination thereof.

19. A method of manufacturing a percutaneous micro implement comprising mounting a micro payload, integrated by cooling coagulation or drying solidification, into an orifice of a stem holder by applying pressure or ultrasonic vibration to form a drug material with thermo plasticity or moist plasticity into a pyramidal or cone shape integrated by a micro mold for a micro head, a micro mold for a micro stem, and a holder.

20. A method of manufacturing a percutaneous micro implement comprising a simultaneous three-dimensional mobile function finely adjustable in the three directions of back and forth, right and left, and up and down and releasing gradually at speed pitch of 0.001 mm per second vertical to a surface of a micro mold, resulting in a fine releasing process of the molds in forming at least one of a micro head and a micro stem.
